(19) United States
(12) Patent Application Publication
(10) Pub. No.: US 2013/0237738 A1
(43) Pub. Date: Sep. 12, 2013

(54) PROCESS FOR PRODUCING AT LEAST ONE OF ETHENE, PROPENE, AND GASOLINE

(71) Applicant: Chad R. Huovie, Park Ridge, IL (US)

(72) Inventor: Chad R. Huovie, Park Ridge, IL (US)

(73) Assignee: UOP, LLC, Des Plaines, IL (US)

(21) Appl. No.: 13/416,547

(22) Filed: Mar. 9, 2012

(51) Int. Cl. C07C 4/06; C10G 11/05; C10G 11/04

(52) U.S. Cl. 585/651; 585/653; 208/122; 208/120.01

(57) ABSTRACT

One exemplary embodiment can be a process for producing at least one of ethene, propene, and gasoline. The process may include reacting a feed boiling above about 340° C. in the presence of a composition including at least about 55%, by weight, alumina. Often, the composition is the sole catalyst utilized in the reaction.

### FIELD OF THE INVENTION

The present invention generally relates to a process for producing at least one of ethene, propene, and gasoline.

### DESCRIPTION OF THE RELATED ART

Typically, a fluid catalytic cracking (hereinafter may be abbreviated “FCC”) unit can be designed for maximum propene production utilizing a conventional FCC catalyst system. Such conventional FCC catalyst systems usually utilize a Y-zeolite with a high concentration of ZSM-5 zeolite. However, these conventional systems may be limited in their ability to shift yield selectivities between ethene, propene, and butene for a given ZSM-5 zeolite concentration level. As such, present systems often do not produce the desired amounts of propene and ethene as compared to butene. As a consequence, there is a desire to increase the yield of ethene and propene in comparison to butene.

### SUMMARY OF THE INVENTION

One exemplary embodiment can be a process for producing at least one of ethene, propene, and gasoline. The process may include reacting a feed boiling above about 340° C. in the presence of a composition including at least about 55%, by weight, alumina. Often, the composition is the sole catalyst utilized in the reaction.

Another exemplary embodiment may be a process for producing at least one of ethene, propene, and gasoline. The process can include reacting a feed boiling above about 340° C. in the presence of a composition including at least about 65%, by weight, alumina and no more than about 30%, by weight silica, and is essentially free of zeolite. Moreover, the composition can include less than about 1%, by weight, reduced metal. Often, the composition is the sole catalyst utilized in the reaction.

Yet a further exemplary embodiment can be a process for producing at least one of ethene, propene, and gasoline. The process can include reacting a feed boiling above about 340° C. in the presence of a composition having at least about 15%, by weight, of a bottoms cracking additive, a ZSM-5 zeolite, and a Y-zeolite.

The embodiments disclosed herein can provide a further conversion via the addition of a separate, non-zeolitic matrix to an FCC catalyst inventory system that can enhance overall production of ethene and propene while reducing the amount of heavier alkenes, such as butene. Generally, the embodiments herein provide a mechanism for managing overall ethene, propene, and butene selectivity via the addition of a non-zeolitic matrix to the FCC catalyst charged system. Thus, the embodiments provided herein can allow the addition of a non-zeolitic material that can produce additional desired lower alkenes, such as ethene and propene.

### DEFINITIONS

As used herein, the term “pore size” can be expressed in terms of a diameter of an opening or a width of a slit. Generally, pores with diameters or slits with widths of less than 20 angstroms can be referred to as micropores; those of about 20- about 500 angstroms can be referred to as mesopores; and those of greater than 500 angstroms can be referred to as macropores. The pore size can be determined by the Barrett-Joyner-Halenda (hereinafter may be abbreviated “BJH”) adsorption average pore diameter algorithm utilized with an accelerated surface area and porosimetry system sold under the trade designation “ASAP 240” by Micromeritics Instrument Corporation of Norcross, Ga.

As used herein, the term “catalyst” can refer to any substance that a small proportion notably affects the rate of a chemical reaction without itself being consumed or undergoing a chemical change.

As used herein, the term “bottoms cracking additive” generally includes a matrix for cracking of heavier hydrocarbons, e.g., one or more C22-C45 hydrocarbons, and excludes a zeolite and no more than 1%, by weight, of a reduced metal content.

As used herein, the term “zeolite” can mean a hydrated silicate of aluminum and at least one of sodium and calcium, and can include one or more substituted rare-earth oxides.

As used herein, the term “matrix” can refer to a non-zeolitic material having one or more pores including a pore size of at least about 20 angstroms. Generally, a matrix contains substantially pores of at least about 20 angstroms. In other words, of all the pores, at least about 90%, or even about 99%, have a pore size of at least about 20 angstroms. The matrix generally includes no more than about 1%, by weight, of any metal, such as Fe, Li, Ni, and V, in a reduced form, but may include mostly metals in an oxide form, such as an alumina, a titania, and a zirconia, and include a silica.

As used herein, the term “sole catalyst” means that a composition is the sole type of catalytic particle utilized in the reaction and no other types of catalytic particles are utilized. As an example, if a composition including about 55%, by weight, alumina is utilized, no other catalytic composition is present to facilitate cracking of the hydrocarbons.

As used herein, the term “essentially free” means that a composition includes no more than about 0.1%, by weight, of the prefaced ingredient. As an example, a composition being essentially free of zeolite means the composition has no more than about 0.1%, by weight, zeolite.

As used herein, the term “matrix additive” includes a matrix often comprising alumina.

As used herein, the terms “ethene” and “ethylene” may be used interchangeably.

As used herein, the terms “propene” and “propylene” may be used interchangeably.

As used herein, the terms “olefins” and “alkenes” may be used interchangeably.

As used herein, the term “butene” can include one or more of 1-butene, cis-2-butene, trans-2-butene, and 2-methylpropene.

As used herein, the term “kilopascal” may be abbreviated “kPa”.

As used herein, the term “gram” may be abbreviated “g”.

As used herein, the term “weight percent” may be abbreviated “wt. %”.

As used herein, the term “conversion” can mean the amount of feed converted based on the original amount of feed. As an example, the weight percent conversion of the feed can be calculated as:

((feed weight)−(product weight boiling above 221° C.))/(feed weight)

As an example, if a feed weight is 100 g and the product weight boiling above 221° C. is 30 g, then the conversion can be calculated as 70%, by weight.

As used herein, the term “yield” can mean the weight percent of a product, such as ethene or propene, based on the weight of the feed. The yield of product, such as ethene, can be calculated as:

(ethene product weight)/(feed weight)*100

As an example, if a feed weight is 100 g and the ethene product weight is 30 g, then the ethene yield can be calculated as 30%, by weight.

As used herein, the term “second order conversion” is a calculation to linearize the conversion percent and may be abbreviated as “2^{ND} Order Conversion”. The second order conversion can be calculated as follows:

(conversion weight percent)/(100%−conversion weight percent)

Thus, if the conversion weight percent of the feed is 80%, by weight, then the second order conversion is 4.

As used herein, the term “selectivity” can be the amount of a product produced relative to conversion. This relationship can be expressed as a percentage of the yield divided by conversion. The selectivity can be calculated as follows:

(yield weight percent)/(conversion weight percent)*100

As an example, the ethene selectivity can be expressed as 25% for an ethene yield of 20%, by weight, and a feed conversion of 80%, by weight.

As used herein, the boiling range distribution of petroleum fractions can be determined by gas chromatography according to ASTM D2887-08.

### BRIEF DESCRIPTION OF THE DRAWINGS

FIG. 1 is a graphical depiction of propene yield versus second order conversion for several runs with various catalytic compositions.

FIG. 2 is a graphical depiction of ethene yield versus second order conversion for several runs using various catalytic compositions.

FIG. 3 is a graphical depiction of butene yield versus second order conversion for several runs using various catalytic compositions.

FIG. 4 is a graphical depiction of ethene selectivity versus propene selectivity for several runs using various catalytic compositions.

FIG. 5 is a graphical depiction of butene selectivity versus propene selectivity for several runs using various catalytic compositions.

FIG. 6 is a graphical depiction of butene selectivity versus ethene selectivity for several runs using various catalytic compositions.

### DETAILED DESCRIPTION

In one exemplary embodiment, a composition can include at least about 55, about 56, about 57, about 58, about 59, about 60, about 65, about 66, about 67, or about 68%, by weight, of a matrix, such as an alumina. In one desired composition, the composition can include at least about 91%, about 95%, about 96%, about 97%, about 98%, about 99%, or about 100%, by weight, of a matrix, such as an alumina

Generally, the matrix can have one or more pores with a pore size of at least about 20 angstroms, and preferably substantially includes pores with a pore size of at least about 20 angstroms. Alternatively, the matrix can have one or more pores with a pore size of at least about 50, about 60, about 70, about 80, about 90, or about 100 angstroms. Often, the matrix includes one or more mesopores with a pore size of about 20- about 500 angstroms, such as about 73- about 98 angstroms, about 73- about 78 angstroms, and about 95- about 98 angstroms. Typically, the matrix can include at least one of a clay, a silica, and a metal oxide. Generally, the clay can include a bentonite and a kaolin. Often, the metal oxide can include at least one of an alumina, a titania, and a zirconia. Typically, the matrix can be a bottoms cracking additive or matrix additive, which can include substantially alumina.

Usually, the matrix can be utilized steamed or unsteamed. If steamed, the matrix may be steamed for any suitable length of time, such as about 4- about 48 hours, preferably about 15- about 24 hours, and at a pressure of about 100- about 500 kPa.

In yet another exemplary embodiment, a composition can include an effective amount of a matrix for providing catalytic activity and optionally one or more zeolites contained within a catalyst system mixture. Thus, the combination can include a matrix and a ZSM-5 zeolite, a Y-zeolite, or a combination thereof. As an example, the zeolites can include a combination of zeolites, such as a combination of a Y-zeolite and ZSM-5 zeolite. Often, such a combination of zeolites is commercially available for use in FCC units. An exemplary composition can include at least about 15%, about 25%, about 30%, or about 50% of bottoms cracking additive or a matrix. In one exemplary embodiment, the composition can include up to about 10%, by weight, ZSM-5 zeolite; or up to about 50% or about 55%, by weight, Y-zeolite; and up to about 38% or even up to about 60%, by weight, matrix. Such ZSM-5 and Y-zeolites are disclosed in, e.g., U.S. Pat. No. 5,554,274.

In another exemplary embodiment, a deactivated ZSM-5 catalyst can be blended with a steam treated bottoms cracking additive to provide the composition with at least about 15, about 25 or about 50%, by weight, bottoms cracking additive. If the composition is a mixture, such as the matrix and ZSM-5 zeolite and optionally Y-zeolite, the material can be slurried, admixed, or combined, optionally with a binder although the matrix may serve as the binder at a pH of about 2- about 12. A suitable ZSM-5 zeolite and/or procedures are disclosed in, e.g., U.S. Pat. No. 5,554,274.

Thus, the embodiments disclosed herein may demonstrate the synergistic effects of using the bottoms cracking additive matrix and high ZSM-5 systems to maximize propene selectivity. The amorphous matrix can be supplied via catalyst supplier. Generally, this matrix can be added to a separate additive injection system. Alternatively, the matrix can be provided via catalyst reformulation where the total amount of matrix can be increased in the catalyst composition.

Although not wanting to be bound by theory, the embodiments described herein can leverage the capability of the bottoms cracking additive matrix to generate ethene and propene via balance of thermal and catalytic mechanisms. When this system is used in conjunction with a high ZSM-5 and conventional FCC catalyst systems, increases in overall ethene and/or propene selectivity may be observed. This concept can enable a refiner to maximize ethene and/or propene yields without increasing the ratio of catalyst to oil.

Generally, the composition disclosed in the embodiments herein can be utilized in various systems for producing ethene, propene, and gasoline. Often, the composition is utilized in fluid catalytic cracking systems with a feed, such as vacuum gas oil, an atmospheric gas oil, or other similar feeds having a boiling point above about 340° C. and optionally including one or more C22-C45 hydrocarbons. Usually, the fluid catalytic cracking systems can utilize any suitable system having a riser reactor, such as U.S. Pat. No. 5,154,818 and U.S. Pat. No. 4,090,948.

This mixture may be run at a ratio of catalyst to oil of about 4- about 9 at about 560° C. A typical vacuum gas oil can be cracked to yield lower chain alkenes, such as ethene, propene, and butene. Although conversion may be reduced with increasing the concentration of bottoms cracking additive, the selectivity to ethene and propene can be increased relative to a mixture consisting of the catalyst absent the bottoms cracking additive. Generally, selectivity to butene can be reduced by increasing the amount of the bottoms cracking additive.

Thus, processes utilizing the compositions disclosed herein can produce products determined by using any suitable method, such as a gas chromatograph in accordance with ASTM D2887-08.

### Illustrative Embodiments

The following examples are intended to further illustrate the subject catalyst. These illustrations of embodiments of the invention are not meant to limit the claims of this invention to the particular details of these examples. These examples are based on engineering calculations and actual operating experience with similar processes. It should be noted that the selectivities depicted in the tables may differ from selectivities calculated from data in the tables by up to about 2% due to rounding.

Two compositions, namely Compositions A and B, are tested for catalytic properties. Composition A is a mixture of ZSM-5 zeolite and Y-zeolite, and Composition B is a bottoms cracking additive. The table below depicts their respective compositions:

TABLE 1

| | Composition A | Composition B |
|---|---|---|
| Component | (wt. %) | (wt. %) |
| Al₂O₃ | 41.09 | 68.03 |
| CaO | 0.07 | 0.13 |
| Co | 0.01 | |
| Cr | 0.01 | 0.01 |
| Fe | 0.60 | 0.41 |
| K₂O | 0.05 | 0.06 |
| Li | | 0.01 |
| MgO | 0.05 | 0.40 |
| Na | 0.37 | 0.09 |
| Ni | 0.10 | |
| P₂O₅ | 2.96 | 0.08 |
| SiO₂ | 52.96 | 29.46 |
| SrO | 0.00 | |
| TiO₂ | 1.63 | 1.31 |
| V | 0.07 | 0.01 |
| ZnO | 0.02 | 0.01 |
| Zr | 0.01 | 0.01 |

The following table provides the average pore diameter for several batches:

TABLE 2

| | | | | | Composition B | |
|---|---|---|---|---|---|---|
| | | | | | Steamed | |
| | | | Composition B | | Batch 1 | Batch 2 |
| | Composition A | | Unsteamed | | (15 hours at | (24 hours at |
| Properties | Batch 1 | Batch 2 | Batch 1 | Batch 2 | 108 kPa) | 108 kPa) |
| BJH | 95 | 96 | 73 | 78 | 95 | 98 |
| Adsorption | | | | | | |
| Average Pore | | | | | | |
| Diameter | | | | | | |
| (Angstrom) | | | | | | |

For determining average pore diameter by BJH Adsorption, each batch sample is prepared by weighing to about 0.250 g and placed on a degas rack. The initial temperature is set to 90° C. and evacuated for about 60 minutes. The temperature is ramped at 10° C. a minute unless the vacuum pressure exceeds 0.067 kPa. If such an increase in vacuum pressure occurs, the heat is shutoff until the pressure drops below 0.067 kPa. Once the pressure is below 0.067 kPa, the heat is turned back on and the temperature is continued to be incrementally increased. After the final preparation temperature of 400° C. is reached, and the temperature is held for 16 hours. Next, a leak check is performed on each sample by isolating and monitoring for any pressure change for 10 minutes not greater than 0.00001 kPa. That being done, the heating mantle is turned off and the sample is allowed to cool to room temperature. After being cooled, the sample is removed from the instrument following the manufacturer's instructions and the sample is weighed to the nearest 0.0001 gram.

After the sample is prepared, the sample is measured for average pore diameter by BJH Adsorption utilizing a device sold under the trade designation “ASAP 240” by Micromeritics Instrument Corporation of Norcross, Ga. The BJH Adsorption is conducted using Harkins and Jura thickness curve with a standard BJH correction. The minimum and maximum BJH width are, respectively, 17 and 3000 angstroms.

These compositions are used in various combinations to create samples tested in several runs depicted in the figures. A vacuum gas oil is utilized as the feed in the runs. Sample 1 utilizes Composition A and is tested in six runs. The following parameters are constant for all six runs (Runs 1-6): a feed flow rate of 1.0 gram per minute; an oil injection time of 60 seconds; a nitrogen flow during reaction of 140 standard cubic centimeters per minute; a feed injector depth of 2.86 centimeters; and a reaction temperature of 560° C. The parameters of the six runs for Sample 1 are as follows:

TABLE 3
Runs for Sample 1

| | Run 1 | Run 2 | Run 3 | Run 4 | Run 5 | Run 6 |
|---|---|---|---|---|---|---|
| Active | 3.0 | 9.0 | 0.0 | 5.0 | 1.0 | 7.0 |
| Catalyst | | | | | | |
| (g) | | | | | | |
| Reaction | 112 | 113 | 112 | 113 | 112 | 113 |
| Pressure | | | | | | |
| (kPa) | | | | | | |
| Normalized | 2.96 | 8.91 | 0.00 | 4.93 | 0.99 | 6.95 |
| Catalyst:Oil | | | | | | |
| Ratio | | | | | | |
| Reactor | 1.37 | 1.26 | 1.48 | 1.32 | 1.44 | 1.30 |
| Residence | | | | | | |
| Time | | | | | | |
| (second) | | | | | | |
| Hydrocarbon | 62.10 | 64.6 | 59.0 | 63.4 | 60.2 | 64.0 |
| Partial | | | | | | |
| Pressure | | | | | | |
| (kPa) | | | | | | |
| Weight | 20.0 | 6.7 | N/A | 12.0 | 60.0 | 8.6 |
| Hourly | | | | | | |
| Space | | | | | | |
| Velocity | | | | | | |
| (hr⁻¹) | | | | | | |

*N/A means data not applicable.

Amounts of ethene and propene from the reaction zone are measured and are input to calculate conversions and yields for the various runs:

TABLE 4
Runs for Sample 1

| | Run 1 | Run 2 | Run 3 | Run 4 | Run 5 | Run 6 |
|---|---|---|---|---|---|---|
| Ethene (g) | 0.017 | 0.020 | 0.030 | 0.018 | 0.022 | 0.019 |
| Propene (g) | 0.097 | 0.137 | 0.036 | 0.120 | 0.059 | 0.132 |
| Butene (g) | 0.100 | 0.122 | 0.029 | 0.114 | 0.062 | 0.119 |
| Product | 38.9 | 22.0 | 61.3 | 29.6 | 53.5 | 24.0 |
| Percent | | | | | | |
| Boiling Above | | | | | | |
| 221° C. | | | | | | |
| Feed | 61.1 | 78.0 | 38.7 | 70.4 | 46.5 | 76.0 |
| Conversion | | | | | | |
| (%, by | | | | | | |
| weight) | | | | | | |
| Feed 2^{ND} | 1.57 | 3.55 | 0.63 | 2.38 | 0.87 | 3.17 |
| Order | | | | | | |
| Conversion | | | | | | |
| Ethene | 2.76 | 2.57 | 7.73 | 2.55 | 4.66 | 2.54 |
| Selectivity | | | | | | |
| Propene | 15.86 | 17.51 | 9.23 | 16.97 | 12.57 | 17.34 |
| Selectivity | | | | | | |
| Butene | 16.32 | 15.63 | 7.58 | 16.23 | 13.24 | 15.65 |
| Selectivity | | | | | | |

Samples 2-4 utilize, respectively, Composition A; 75%, by weight, of Composition A and 25%, by weight, of steamed Composition B; and 50%, by weight, of Composition A and 50%, by weight, of steamed Composition B; and each of these samples has two runs for a total six runs. The following parameters are constant for all six runs: a feed flow rate of 1.0 gram per minute; an oil injection time of 60 seconds; a nitrogen flow during reaction of 140 standard cubic centimeters per minute; a feed injector depth of 2.86 centimeters; and a reaction temperature of 560° C. The six runs for Sample 2-4 are as follows:

TABLE 5
Runs for Samples 2-4

| | Sample 2 | | Sample 3 | | Sample 4 | |
|---|---|---|---|---|---|---|
| | Run 1 | Run 2 | Run 1 | Run 2 | Run 1 | Run 2 |
| Active | 9 | 4 | 4 | 9 | 4 | 9 |
| Catalyst | | | | | | |
| (g) | | | | | | |
| Reaction | 114 | 112 | 113 | 114 | 113 | 116 |
| Pressure | | | | | | |
| (kPa) | | | | | | |
| Normalized | 8.87 | 4.01 | 3.99 | 8.96 | 4.00 | 8.91 |
| Catalyst:Oil | | | | | | |
| Ratio | | | | | | |
| Reactor | 1.27 | 1.35 | 1.35 | 1.28 | 1.36 | 1.30 |
| Residence | | | | | | |
| Time | | | | | | |
| (second) | | | | | | |
| Hydrocarbon | 65.4 | 62.1 | 62.9 | 65.4 | 62.9 | 65.8 |
| Partial | | | | | | |
| Pressure | | | | | | |
| (kPa) | | | | | | |
| Weight | 6.7 | 15.0 | 15.0 | 6.7 | 15.0 | 6.7 |
| Hourly | | | | | | |
| Space | | | | | | |
| Velocity | | | | | | |
| (hr⁻¹) | | | | | | |

Amounts of ethene and propene from the reaction zone are measured and are input to calculate conversions and yields for the various runs:

TABLE 6
Runs for Samples 2-4

| | Sample 2 | | Sample 3 | | Sample 4 | |
|---|---|---|---|---|---|---|
| | Run 1 | Run 2 | Run 1 | Run 2 | Run 1 | Run 2 |
| Ethene (g) | 0.021 | 0.018 | 0.018 | 0.022 | 0.019 | 0.022 |
| Propene (g) | 0.138 | 0.111 | 0.107 | 0.138 | 0.099 | 0.134 |
| Butene (g) | 0.119 | 0.111 | 0.109 | 0.123 | 0.101 | 0.118 |
| Product | 21.6 | 31.7 | 34.3 | 22.1 | 36.3 | 25.5 |
| Percent | | | | | | |
| Boiling Above | | | | | | |
| 221° C. | | | | | | |
| Feed | 78.4 | 68.3 | 65.7 | 77.9 | 63.7 | 74.5 |
| Conversion | | | | | | |
| (%, by | | | | | | |
| weight) | | | | | | |
| Feed 2^{ND} | 3.64 | 2.15 | 1.92 | 3.52 | 1.75 | 2.92 |
| Order | | | | | | |
| Conversion | | | | | | |
| Ethene | 2.64 | 2.59 | 2.77 | 2.76 | 3.02 | 2.99 |
| Selectivity | | | | | | |
| Propene | 17.57 | 16.21 | 16.21 | 17.78 | 15.60 | 17.95 |
| Selectivity | | | | | | |
| Butene | 15.17 | 16.33 | 16.58 | 15.74 | 15.85 | 15.88 |
| Selectivity | | | | | | |

Sample 5 utilizes unsteamed Composition B and data from six runs. The following parameters are constant for all six runs (Runs 1-6): a feed flow rate of 1.0 gram per minute; an oil injection time of 60 seconds; a nitrogen flow during reaction of 140 standard cubic centimeters per minute; a feed injector depth of 2.86 centimeters; and a reaction temperature of 560° C. Data from the six runs for Sample 5 are as follows:

TABLE 7
Runs for Sample 5

| | Run 1 | Run 2 | Run 3 | Run 4 | Run 5 | Run 6 |
|---|---|---|---|---|---|---|
| Active | 3.0 | 9.0 | 0.0 | 5.0 | 1.0 | 7.0 |
| Catalyst | | | | | | |
| (g) | | | | | | |
| Reaction | 113 | 123 | 112 | 117 | 113 | 120 |
| Pressure | | | | | | |
| (kPa) | | | | | | |
| Normalized | 2.95 | 8.83 | 0.00 | 4.90 | 1.03 | 6.87 |
| Catalyst:Oil | | | | | | |
| Ratio | | | | | | |
| Reactor | 1.35 | 1.34 | 1.48 | 1.35 | 1.44 | 1.35 |
| Residence | | | | | | |
| Time | | | | | | |
| (second) | | | | | | |
| Hydrocarbon | 63.0 | 70.9 | 59.2 | 65.9 | 60.3 | 68.8 |
| Partial | | | | | | |
| Pressure | | | | | | |
| (kPa) | | | | | | |
| Weight | 20.0 | 6.7 | N/A | 12.0 | 60.0 | 8.6 |
| Hourly | | | | | | |
| Space | | | | | | |
| Velocity | | | | | | |
| (hr⁻¹) | | | | | | |

*N/A means data not applicable.

Amounts of ethene and propene from the reaction zone are measured and are input to calculate conversions and yields for the various runs:

TABLE 8
Runs for Sample 5

| | Run 1 | Run 2 | Run 3 | Run 4 | Run 5 | Run 6 |
|---|---|---|---|---|---|---|
| Ethene (g) | 0.027 | 0.035 | 0.029 | 0.029 | 0.028 | 0.033 |
| Propene (g) | 0.070 | 0.111 | 0.037 | 0.089 | 0.050 | 0.100 |
| Butene (g) | 0.056 | 0.085 | 0.031 | 0.071 | 0.042 | 0.075 |
| Product | 51.8 | 41.0 | 61.1 | 47.6 | 57.1 | 44.6 |
| Percent | | | | | | |
| Boiling Above | | | | | | |
| 221° C. | | | | | | |
| Feed | 48.2 | 59.0 | 38.9 | 52.4 | 42.9 | 55.4 |
| Conversion | | | | | | |
| (%, by | | | | | | |
| weight) | | | | | | |
| Feed 2^{ND} | 0.93 | 1.44 | 0.64 | 1.10 | 0.75 | 1.24 |
| Order | | | | | | |
| Conversion | | | | | | |
| Ethene | 5.50 | 5.94 | 7.54 | 5.54 | 6.54 | 5.87 |
| Selectivity | | | | | | |
| Propene | 14.58 | 18.88 | 9.54 | 16.97 | 11.60 | 18.04 |
| Selectivity | | | | | | |
| Butene | 11.69 | 14.35 | 8.07 | 13.48 | 9.77 | 13.58 |
| Selectivity | | | | | | |

Samples 6-8 utilize, respectively, Composition A; 75%, by weight of Composition A and 25%, by weight, of unsteamed Composition B; and 50%, by weight, of Composition A and 50%, by weight of unsteamed Composition B; and each of these samples has two runs for a total six runs. The following parameters are constant for all six runs: a feed flow rate of 1.0 gram per minute; an oil injection time of 30 seconds; a nitrogen flow during reaction of 140 standard cubic centimeters per minute; a feed injector depth of 2.86 centimeters; and a reaction temperature of 560° C. Data from the six runs for Sample 6-8 are as follows:

TABLE 9
Runs for Samples 6-8

| | Sample 6 | | Sample 7 | | Sample 8 | |
|---|---|---|---|---|---|---|
| | Run 1 | Run 2 | Run 1 | Run 2 | Run 1 | Run 2 |
| Active | 9 | 4 | 4 | 9 | 4 | 9 |
| Catalyst | | | | | | |
| (g) | | | | | | |
| Reaction | 133 | 130 | 132 | 140 | 132 | 139 |
| Pressure | | | | | | |
| (kPa) | | | | | | |
| Normalized | 9.01 | 4.05 | 4.06 | 8.96 | 4.05 | 8.86 |
| Catalyst:Oil | | | | | | |
| Ratio | | | | | | |
| Reactor | 1.51 | 1.57 | 1.61 | 1.57 | 1.61 | 1.56 |
| Residence | | | | | | |
| Time | | | | | | |
| (second) | | | | | | |
| Hydrocarbon | 76.5 | 72.3 | 73.6 | 80.4 | 73.4 | 80.5 |
| Partial | | | | | | |
| Pressure | | | | | | |
| (kPa) | | | | | | |
| Weight | 6.7 | 15.0 | 15.0 | 6.7 | 15.0 | 6.7 |
| Hourly | | | | | | |
| Space | | | | | | |
| Velocity | | | | | | |
| (hr⁻¹) | | | | | | |

Amounts of ethene and propene from the reaction zone are measured and are input to calculate conversions and yields for the various runs:

TABLE 10
Runs for Samples 6-8

| | Sample 6 | | Sample 7 | | Sample 8 | |
|---|---|---|---|---|---|---|
| | Run 1 | Run 2 | Run 1 | Run 2 | Run 1 | Run 2 |
| Ethene (g) | 0.029 | 0.023 | 0.022 | 0.029 | 0.022 | 0.027 |
| Propene (g) | 0.159 | 0.139 | 0.129 | 0.160 | 0.118 | 0.153 |
| Butene (g) | 0.127 | 0.124 | 0.115 | 0.133 | 0.112 | 0.135 |
| Product | 16.8 | 27.6 | 31.5 | 18.5 | 33.2 | 20.6 |
| Percent | | | | | | |
| Boiling Above | | | | | | |
| 221° C. | | | | | | |
| Feed | 83.2 | 72.4 | 68.5 | 81.5 | 66.8 | 79.4 |
| Conversion | | | | | | |
| (%, by | | | | | | |
| weight) | | | | | | |
| Feed 2^{ND} | 4.95 | 2.62 | 2.17 | 4.41 | 2.01 | 3.84 |
| Order | | | | | | |
| Conversion | | | | | | |
| Ethene | 3.52 | 3.19 | 3.23 | 3.58 | 3.25 | 3.43 |
| Selectivity | | | | | | |
| Propene | 19.05 | 19.17 | 18.90 | 19.69 | 17.65 | 19.34 |
| Selectivity | | | | | | |
| Butene | 15.29 | 17.14 | 16.81 | 16.33 | 16.84 | 17.05 |
| Selectivity | | | | | | |

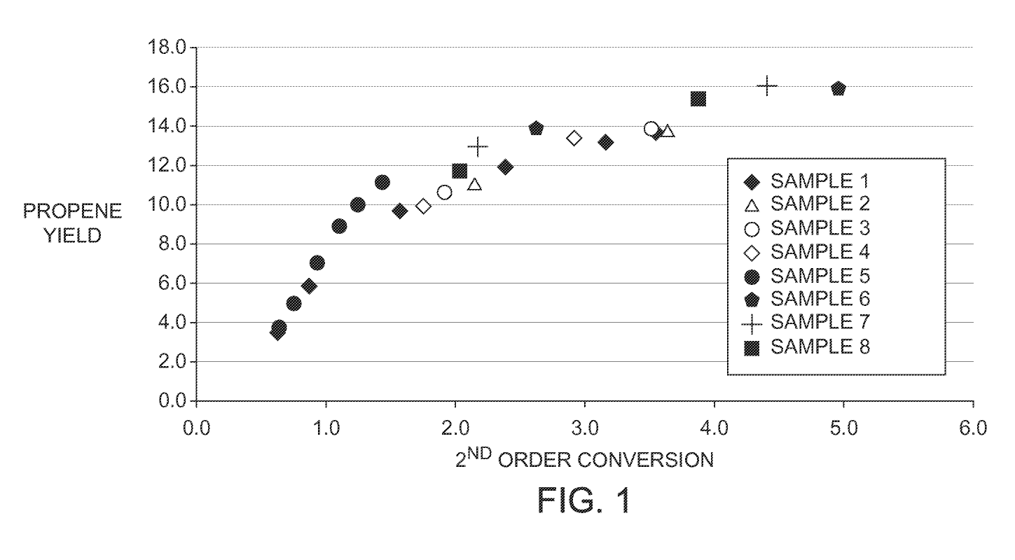

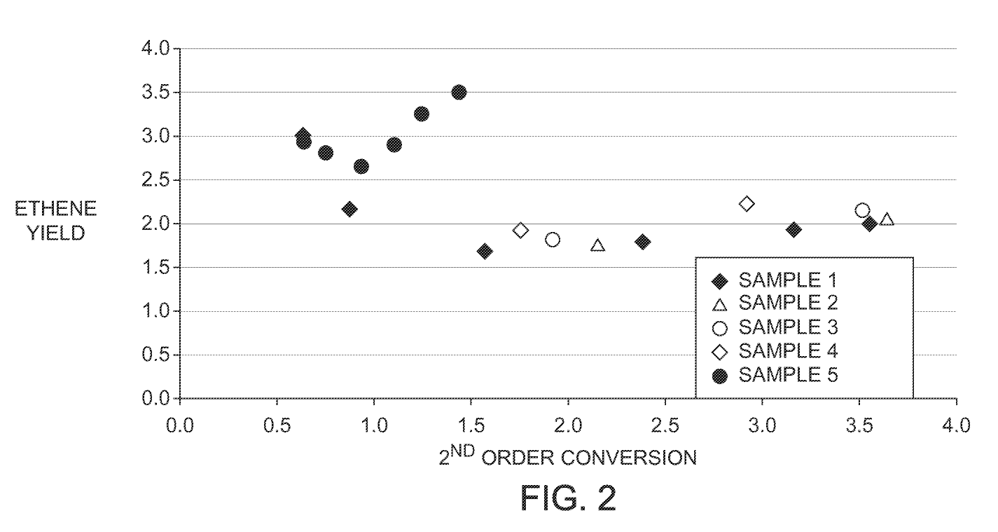

Referring to FIGS. 1 and 2, respectively, the eight samples are plotted with a comparison of propene yield versus second order conversion, and ethene yield versus second order conversion. At a lower second order conversion, Sample 5 with Composition B has a comparable propene yield and a higher ethene yield as compared to other compositions used in other sample runs.

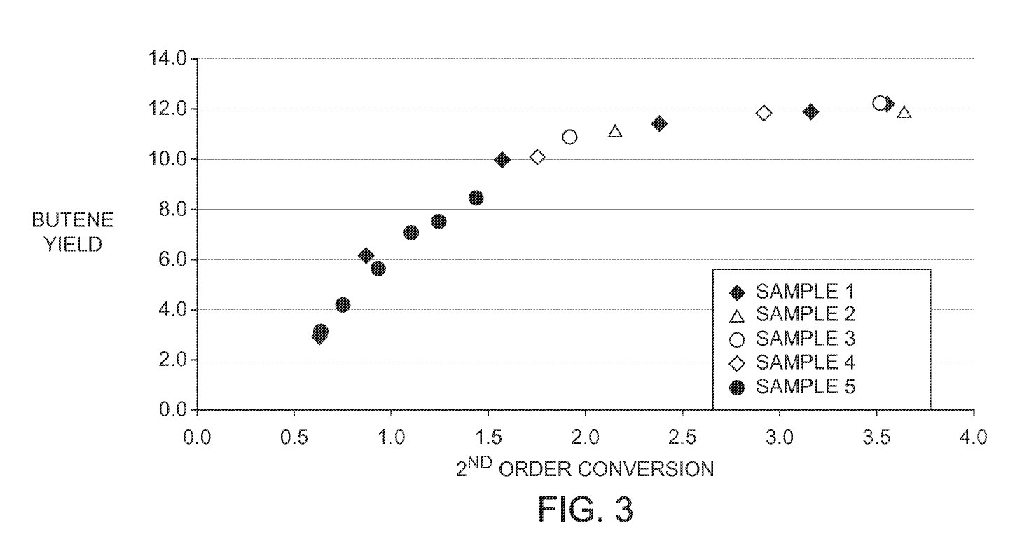

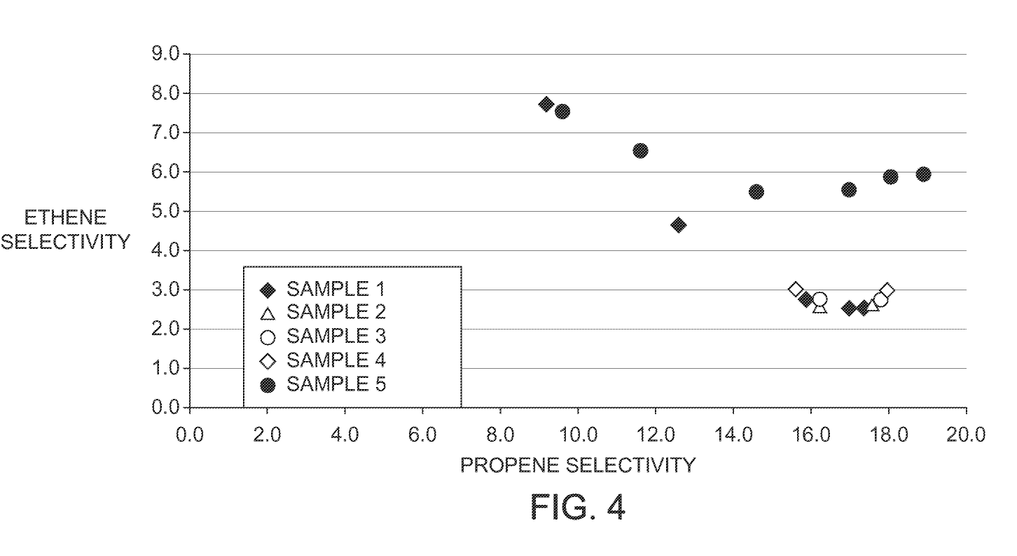

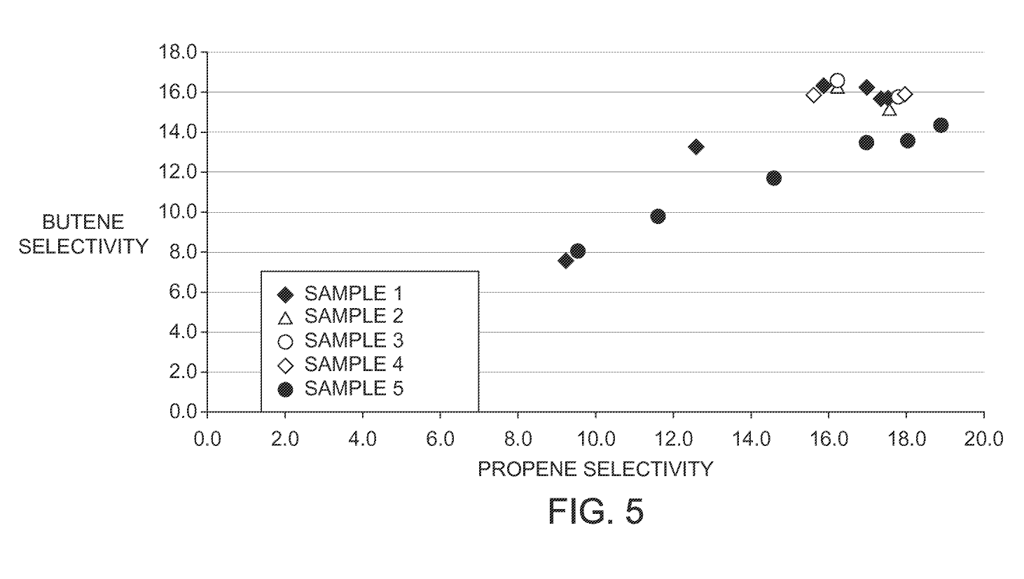

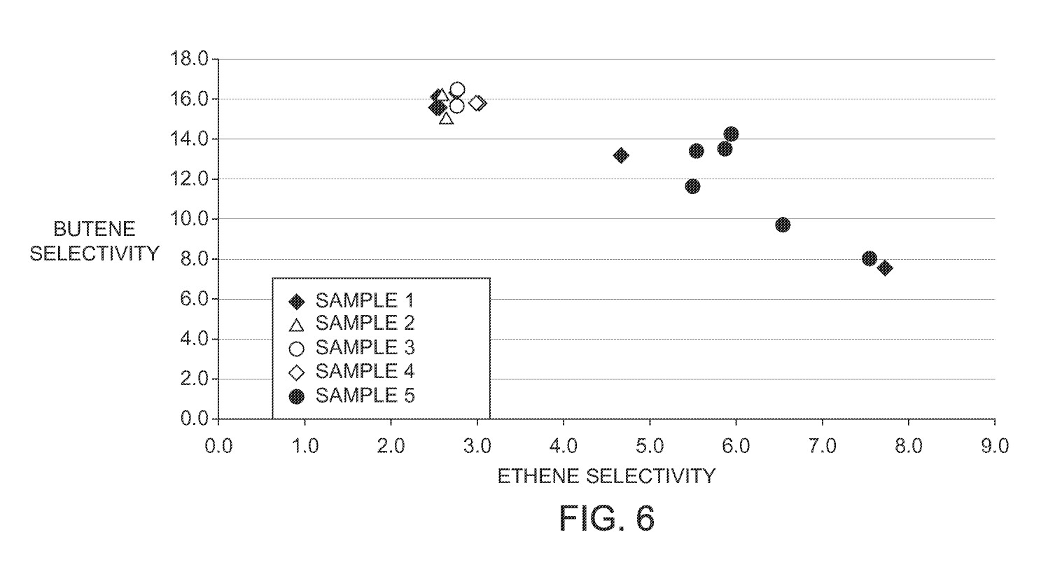

With respect to FIG. 3, higher yields attributed to Composition B result in lower butene yields and correspondingly higher propene and ethene yields. With respect to FIG. 4, comparing ethene selectivity versus propene demonstrates a greater selectivity for ethene for Sample 5 as compared to the other samples. As an aside, the data point for the sixth run of Sample 1 is obscured by the other data points on the plot. With respect to FIGS. 5-6, Sample 5 depicts lower butene selectivity as compared to the other samples with, respectively, propene and ethene. Moreover, the embodiments disclosed herein can replace Composition A, which tends to be more expensive, with Composition B at 25%, or even 50%, by weight, and still obtain comparable results versus using Composition A alone. Not only obtaining better results is unexpected, but similar results are significant as Composition B is generally less expensive than Composition A.

Thus, significant cracking selectivity to propene can be achieved when a standard gas oil is cracked over 100% amorphous matrix material. Typically, propene yields an excess of about 10%, by weight, with a catalyst to oil weight ratio of 9:1, a temperature of about 560° C., and utilizing a feed of vacuum gas oil, can be achieved without the use of a separate zeolitic catalyst in the FCC charge to the reactor. Additionally, it has been found that the selectivity to ethene is also enhanced as increases in ethene:propene ratios are seen relative to a commercially-operated high content ZSM-5 zeolite and Y-zeolite system. The increase in ethene and propene selectivity comes at the expense of butenes, as butene:propene ratios are reduced with the addition of the matrix.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A catalytic cracking process for producing at least one of ethene, propene, and gasoline, comprising:
reacting a feed boiling above about 340° C. in the presence of a composition comprising at least about 55%, by weight, alumina wherein the composition is the sole catalyst utilized in the reaction, wherein the composition comprises an average pore diameter of at least about 73 angstroms, wherein the composition increases the ethene and/or propene selectivity at the expense of butenes.

2. The process according to claim 1, wherein the composition comprises no more than about 30%, by weight, silica.

3. The process according to claim 1, wherein the composition comprises at least about 60%, by weight, alumina and no more than about 30%, by weight, silica.

4. The process according to claim 1, wherein the composition comprises at least about 91%, by weight, alumina.

5. The process according to claim 1, wherein the composition comprises at least about 95%, by weight, alumina.

6. The process according to claim 1, wherein the composition comprises at least about 99%, by weight, alumina.

7. The process according to claim 1, wherein the composition comprises about 100%, by weight, alumina.

8. (canceled)

9. The process according to claim 1, wherein the composition is essentially free of zeolite and comprises less than about 1%, by weight, reduced metal.

10. A catalytic cracking process for producing at least one of ethene, propene, and gasoline, comprising:
reacting a feed boiling above about 340° C. in the presence of a composition comprising at least about 65%, by weight, alumina and no more than about 30%, by weight silica, and is essentially free of zeolite and comprises less than about 1%, by weight, reduced metal, wherein the composition is the sole catalyst utilized in the reaction, wherein the composition comprises an average pore diameter of at least about 73 angstroms, wherein the composition increases the ethene and/or propene selectivity at the expense of butenes.

11-12. (canceled)

13. A catalytic cracking process for producing at least one of ethene, propene, and gasoline, comprising:
reacting a feed boiling above about 340° C. in the presence of a composition consisting essentially of a ZSM-5 zeolite, a Y zeolite and at least about 15%, by weight, of a bottoms cracking additive.

14. The process according to claim 13, wherein the bottoms cracking additive comprises a matrix, and the matrix, in turn, comprises at least one of a clay, a silica, and a metal oxide.

15. The process according to claim 14, wherein the matrix comprises a clay, and the clay, in turn, comprises at least one of a bentonite and a kaolin.

16. The process according to claim 13, wherein the composition comprises at least about 50%, by weight, matrix.

17. The process according claim 14, wherein the matrix comprises substantially pores of at least about 73 to about 98 angstroms.

18. The process according to claim 14, wherein the matrix comprises a metal oxide, wherein the metal oxide comprises at least one of an alumina, a titania, and a zirconia.

19. The process according to claim 13, wherein the composition comprises up to about 10%, by weight, ZSM-5 zeolite.

20. The process according to claim 13, wherein the composition comprises up to about 55%, by weight, Y-zeolite.
